(19) United States
(12) Patent Application Publication
(10) Pub. No.: US 2025/0313784 A1
(43) Pub. Date: Oct. 9, 2025

(54) BIOREACTOR AND METHOD FOR THE PRODUCTION OF CULTURED FAT

(71) Applicant: MOSA MEAT B.V., Maastricht (NL)

(72) Inventors: Nik MAZARI, Maastricht (NL); Jonathan Jan BREEMHAAR, Maastricht (NL); Nick VAN ZOMEREN, Maastricht (NL); Kelly Cornelia Johanna Helena AARTS, Maastricht (NL); Jos SCHEEPERS, Maastricht (NL); Roos KAMPS, Maastricht (NL)

(73) Assignee: MOSA MEAT B.V., Maastricht (NL)

(21) Appl. No.: 18/865,117

(22) PCT Filed: May 17, 2023
(86) PCT No.: PCT/NL2023/050281

§ 371 Date: Nov. 12, 2024

(30) Foreign Application Priority Data
May 18, 2022 (NL) 2031922

(51) Int. Cl. C12M 3/00; C12M 1/00; C12M 1/34; C12N 5/0775

(52) U.S. Cl. CPC C12M 21/08; C12M 29/00; C12M 41/32; C12N 5/0667

(57) ABSTRACT

A method of forming a hydrogel fibre inside a bioreactor is provided. The method comprises steps of providing a container vessel of the bioreactor with a volume of cross-linking fluid therein, and injecting a flow of hydrogel precursor fluid into the cross-linking fluid, whereby the hydrogel fibre is formed, wherein a leading end of the hydrogel fibre revolves at least partially around a vertical axis, in particular a centreline of the container vessel. Bioreactors and bioreactor assemblies for cultivating fat are also provided. The hydrogel fibre is preferably formed from a cell-containing hydrogel precursor fluid comprising proliferated cells, in particular fibro-adipogenic progenitors, in particular of mammalian origin, such as of bovine origin.

### TECHNICAL FIELD

The aspects and embodiments thereof relate to methods and devices for the production of hydrogel fibres, which in particular are used in the production of cultured fat.

### BACKGROUND

Since ancient times, meat has been a major source of high-quality protein in the human diet, and to this day it continues to provide nutrition to the exponentially growing population of the world. However, global meat production has increased so much that it is now one of the largest contributors to a number of serious problems such as animal welfare, pollution, climate change and food safety issues.

The ideal replacement for animal meat would be meat produced through tissue engineering. Virtually all downsides of meat production would be eradicated but the consumers could still enjoy the meat.

For many types of meat, fat is an important component in terms of taste and texture of the meat. As such, when producing cultured meat, such as cultured beef, it may be preferred to provide a mixture of muscle fibres and fat.

### SUMMARY

Present methods and devices only allow for very small quantities—for example in the order of grams—of cultured fat to be produced. In the production of cultured fat, hydrogel scaffolds are used in which proliferated cells are allowed to differentiate when subjected to a differentiation medium. The present methods and devices often require manual actions, such as manual injection of cell-containing hydrogel precursor fluid into a cross-linking fluid, for example using a syringe, and/or require moving the hydrogel scaffolds between different vessels between the time of forming the hydrogel scaffolds and later allowing differentiation of the cells in the hydrogel scaffolds. It is desired to be able to produce larger quantities of fat in relatively less time and/or with relatively less resources and/or with relatively less human effort.

A first aspect provides a method of forming a hydrogel fibre inside a bioreactor. The method comprises steps of providing a container vessel of the bioreactor with a volume of cross-linking fluid therein, and injecting a flow of hydrogel precursor fluid into the cross-linking fluid, whereby the hydrogel fibre is formed.

Preferably, the hydrogel precursor fluid is a cell-containing hydrogel precursor fluid. However, embodiments are also envisioned wherein the hydrogel precursor fluid is essentially free of cells, in particular essentially free of proliferated cells. In these embodiments, the hydrogel fibre formed may be seeded with cells after the fibre has been formed. It will thus be understood that throughout the present disclosure, wherever a cell-containing hydrogel fibre is mentioned, a hydrogel fibre which is essentially free of cells, in particular of proliferated cells, is also envisioned. Similarly, it will thus be understood that throughout the present disclosure, wherever a cell-containing hydrogel precursor fluid is mentioned, a hydrogel precursor fluid which is essentially free of cells, in particular of proliferated cells, is also envisioned.

A leading end of the hydrogel fibre revolves at least partially around a vertical axis, in particular a centreline of the container vessel. By allowing the leading end of the hydrogel fibre to revolve at least partially around the vertical axis, entangling of the fibre may be prevented, or at least a chance of entangling and/or the amount of entanglement may be reduced. Preferably, the leading end of the hydrogel fibre revolves at least partially around the vertical axis during injection of the flow of hydrogel precursor fluid into the cross-linking fluid.

In general, in the present disclosure, the cell-containing hydrogel fibre may also be referred to as hydrogel fibre, or even fibre, for reasons of conciseness. The hydrogel fibre acts as a scaffold for the cells contained in the hydrogel. The fibre may be regarded as a dispersed phase, and the crosslinking fluid may be regarded as a continuous phase.

A cross-sectional shape of the hydrogel may be defined by a cross-sectional shape of an outlet end of an injector, such as an injector needle, through which the flow of cell-containing hydrogel precursor fluid is injected into the cross-linking fluid. This cross-sectional shape defines two orthogonal dimensions of the fibre, which dimensions may be limited due to the biological requirements of the differentiation process of the cells. For example, at least one of these two dimensions may be restricted by a maximum path the nutrients from a differentiation medium can travel through the hydrogel towards cells inside the hydrogel. When the two dimensions defining the cross-sectional shape are equal, the cross-sectional shape may resemble a circle. In general, a circular cross-sectional shape may be preferred. A third dimension of the fibre—a length orthogonal to the cross-section—may be only restricted by the maximum volume available inside the bioreactor.

As the leading end of the hydrogel fibre revolves at least partially around a vertical axis, in particular a centreline of the container vessel, entanglement and/or self-adhesion of the fibre is prevented, or at least reduced. As such, more outer surface area of the fibre may be exposed to fluid in the container vessel, and/or more flow paths past the fibre may be available, for example in a later stage of perfusion.

The leading end of the hydrogel fibre revolving at least partially around the vertical axis in general may imply that the leading end revolves at least through a 45 degrees section of a circumference/arc around said vertical axis, at least 90 degrees, at least 180 degrees, at least 270 degrees, or even 360 degrees or more. A path over which the leading end of the hydrogel fibre revolves may be circular, approximately circular, curved, partially straight, or any combination thereof, wherein the path may be formed by a plurality of differently shaped sections.

By virtue of the method according to the first aspect, a fibre may obtained which is revolved at least partially around the vertical axis. It will be understood that this final shape of the fibre, for example after the flow of hydrogel precursor fluid has stopped, may have any number of connected segments with any shape, for example curved, straight, looped over itself, in any combination thereof, which segments may have any orientation. The final average shape of the fibre may be generally circular around the vertical axis, for example generally shaped as an arc of at least 180 degrees around the vertical axis, or even 270 degrees or more around the vertical axis.

As an additional or alternative option, the flow of cell-containing hydrogel precursor fluid may be injected into the cross-linking fluid generally along an inner wall of the container vessel, which may result in the leading end of the hydrogel fibre being moved in the cross-linking fluid in a direction comprising an azimuthal direction component, in particular to an inside wall of the container vessel. The leading end of the hydrogel fibre may thus move over a path generally following a curvature of the inner wall of the container vessel. The inner wall, or at least part thereof, may be generally cylindrically shaped, or more generally curved in shape.

In general, the azimuthal direction may be regarded in a cylindrical coordinate system, where the longitudinal or axial axis is parallel to a centreline of a container vessel—in particular to an internal volume thereof—and the radial axis or polar axis is regarded orthogonal to the longitudinal axis. The direction of the leading end of the fibre may for at least part of the flow path of the leading end comprise an azimuthal direction component. In particular, the azimuthal direction component may be dominant compared to a radial or polar direction component and/or a longitudinal or axial direction component.

The flow of cell-containing hydrogel precursor fluid may be injected into the cross-linking fluid in a generally horizontal direction. A generally horizontal direction may be defined as being within a +−45 degrees range relative to horizontal, more in particular +−20 degrees range relative to horizontal, within a +−10 degrees range relative to horizontal, or even within a +−5 or +−2 degrees range relative to horizontal.

For revolving the leading end of the hydrogel fibre at least partially around the vertical axis, as a particular option, the flow of cell-containing hydrogel precursor fluid into the cross-linking fluid may be directed at least partially in an azimuthal direction relative to the container vessel, in particular to an inner wall thereof, for example by virtue of an orientation of an injection nozzle through which the flow of cell-containing hydrogel precursor fluid is provided.

Additionally, or alternatively, for revolving the leading end of the hydrogel fibre at least partially around the vertical axis, embodiments of the method may comprise a step of inducing a rotating motion in the cross-linking fluid prior to or during the injecting of the flow of cell-containing hydrogel precursor fluid into the cross-linking fluid. The rotation of the cross-linking fluid may cause the cross-linking fluid to revolve around a generally vertical axis, which axis may in particular correspond with a centreline of the container vessel. The rotating motion may in examples even create a vortex in the cross-linking fluid, or at least a swirling motion in the cross-linking fluid around a substantially vertical axis.

When the flow of cell-containing hydrogel precursor fluid is injected into the rotating cross-linking fluid, an azimuthal or circumferential direction component may be added to the flow direction of the hydrogel fibre formed by the cell-containing hydrogel precursor fluid reacting with the cross-linking fluid.

In general, a rotating motion may be induced in the cross-linking fluid by virtue of a rotating element submerged in the cross-linking fluid, such as a rotor, for example an impeller or a propeller comprising one or more vanes and/or blades.

Additionally, or alternatively, a rotating motion may be induced in the cross-linking fluid by virtue of injecting a flow of fluid into the cross-linking fluid in a generally azimuthal direction. In general, it will be understood that a fluid may comprise one or more gasses and/or one or more liquids, in any combination thereof. In certain embodiments, the flow of fluid injected to induce the rotating motion may be or comprise water, air, or cross-linking fluid. The flow of fluid may be injected into the cross-linking fluid using a separate injector, or the same injector as used for injecting the flow of cell-containing hydrogel precursor fluid into the cross-linking fluid.

For example in order to increase a production capacity of a bioreactor, as a particular option, embodiments of the method according to the first aspect may comprise a step of injecting any number of additional flows of cell-containing hydrogel precursor fluid into the cross-linking fluid in the container vessel of the bioreactor, whereby one or more additional cell-containing hydrogel fibres may be formed, wherein the cell-containing hydrogel fibre and the additional cell-containing hydrogel fibre or fibres are preferably separated by one or more separators of the bioreactor. Multiple flows of cell-containing hydrogel precursor fluid may at least in part be injected into the cross-linking fluid simultaneously, for example at different locations in the cross-linking fluid.

When an injection needle is moveable within the internal volume of the container vessel, multiple distinct fibres may be formed at multiple locations inside the internal volume. Additionally, or alternatively, when an injection needle is moveable within the internal volume of the container vessel, the needle may be moved while injecting a flow of cell-containing hydrogel precursor fluid into the container vessel using said injection needle. For example, the injection needle may be rotated about a substantially vertical axis, translated in a horizontal plane, translated in a substantially vertical direction, translated in any other direction, rotated about any other rotation axis, or any combination thereof.

As an option for injecting the flow of cell-containing hydrogel precursor fluid into the cross-linking fluid, a pressurised gas may be used. In particular, when the cell-containing hydrogel precursor fluid is supplied from a fluid container, the cell-containing hydrogel precursor fluid may be forced out of the fluid container by injecting the pressurised gas into the fluid container. In general, the term pressurised gas may refer to a gas which is pressurised to any pressure above ambient pressure.

In general, the flow of cell-containing hydrogel precursor fluid injected into the cross-linking fluid may be supplied through a fluid conduit. Embodiments of the method according to the first aspect may further comprise flushing at least a part of the fluid conduit, in particular a downstream part ending inside the bioreactor, with a flushing fluid prior to injecting the cell-containing hydrogel precursor fluid into the cross-linking fluid. By flushing the fluid conduit prior to injecting the cell-containing hydrogel precursor fluid into the cross-linking fluid, it may be prevented that cross-linking fluid flows into the fluid container holding the cell-containing hydrogel precursor fluid via the fluid conduit and/or into the fluid conduit.

In general, a conduit, such as a fluid conduit or a gas conduit, may comprise one or more hoses, pipes, channels, or any other conduit through which a fluid may be transported. It will be understood that the fluid conduit may comprise any number of interconnected conduits. For example, a fluid conduit may comprise a length of stainless steel pipe and/or flexible tubing.

As a particular option, pressurised gas from a single source of pressurised gas, such as a gas cylinder or compressor, may be used both for flushing the fluid conduit as well as for forcing cell-containing hydrogel precursor fluid out of the fluid container. The pressurised gas may for example be or comprise carbon dioxide, air, an inert gas, or any other gas which preferably does not chemically react with the cross-linking fluid. Alternatively, different sources of pressurised gas may be used for flushing the fluid conduit and for forcing cell-containing hydrogel precursor fluid out of the fluid container.

It has been observed that cell-containing hydrogel fibres may be fragile and may hence be prone to breaking if subjected to even relatively small forces, such as their own weight when suspended in the air. As such, it may be preferred to have the cell-containing hydrogel fibres floating in a liquid to prevent breaking the delicate structure of the fibres, also when the cells inside the cell-containing hydrogel fibres are differentiating after being exposed to a differentiation medium. The differentiation medium may then be the liquid in which the cell-containing hydrogel fibres are allowed to float.

To this end, a second aspect provides a method of cultivating fat, wherein preferably one or more cell-containing hydrogel fibres are kept submerged in a liquid between forming of the hydrogel fibre and throughout at least part of a differentiation process of cells in the one or more cell-containing hydrogel fibres.

The method according to the second aspect comprises steps of forming a cell-containing hydrogel fibre inside a bioreactor, in particular using any embodiment of the method according to the first aspect, replacing the cross-linking fluid in the container vessel with a differentiation medium, allowing differentiation of cells in the cell-containing hydrogel fibre into fat, and removing the fat from the container vessel.

By virtue of the method according to the second aspect, a need to move one or more of the fragile cell-containing hydrogel fibres, for example between different vessels, may be eliminated or at least reduced, in particular between forming of the hydrogel fibres and at least part of the time required for differentiation of cells in the cell-containing hydrogel fibre into fat.

After at least some of the cells in the cell-containing hydrogel fibre have differentiated into fat, the hydrogel fibre may be referred to as a fat-containing hydrogel fibre. Fat may be trapped inside the hydrogel.

Different ways of removing the fat from the container vessel are envisioned. For example, the fat-containing hydrogel fibre may be subjected to shear forces, which results in the fibre breaking into smaller segments. These smaller segments may be flushed from the container vessel in order to remove the fat from the container vessel. To break the fibre into smaller segments, for example the liquid in which the fibre is held may be agitated, for example using one or more impellers or other rotating element or rotating motion inducer. In particular when the bioreactor comprises a separator, impellers may be positioned above and below said separator to improve agitation of the fluid inside the container vessel.

Another example of a way of removing the fat from the container vessel is to provide a high shear flow passing through the internal volume of the container vessel. By virtue of the high shear flow, the fat-containing fibre may break down into smaller segment, which segments are caught in the high shear flow and may be filtered out of the high shear flow, for example using a filter or sieve positioned outside the container vessel or at least outside the internal volume of the container vessel. As another example, fat may be removed from the container vessel through one or more sampling ports comprised by the container vessel, which sampling ports allow access into the internal volume of the container vessel, for example through a side wall of the container vessel. As yet another option, the internal volume may be flushed with sodium citrate or any other compound in which the hydrogel fibre can be dissolved, such that the fat is released from the hydrogel.

The differentiation medium may be transported past the one or more cell-containing hydrogel fibres. This may allow for improved transfer of nutrients required to promote cells to differentiate. These nutrients are contained in the differentiation medium.

Replacing the cross-linking fluid in the container vessel with the differentiation medium may imply that at a first instance in time, the container comprises or contains cross-linking fluid, at that at a second instance in time, after the first instance in time, the container comprises or contains differentiation medium. In general, it will be understood that between the first instance in time and the second instance in time, the container may comprise any other fluid, and may be essentially free of cross-linking fluid and/or differentiation medium.

For example, as a particular option, embodiments of the method for cultivating fat may further comprise replacing the cross-linking fluid in the container vessel with a basal medium, and subsequently replacing the basal medium with the differentiation medium.

The basal medium may comprise any combination of one or more sugars, one or more salts, and/or one or more amino acids. The basal medium may be used to wash away the cross-linking fluid from the container vessel. Examples of basal medium are Minimal Essential Medium, DMEM (Dulbecco's Modified Eagle's Medium and Basal Medium Eagle (BME).

In general, one or more fluids, such as a cross-linking fluid, basal medium, and differentiation medium, may be pumped, poured, or otherwise transported into the container vessel, for example using one or more pumps or pressurisation systems. Furthermore, one or more fluids present in the container vessel may be removed from said container vessel, for example using one or more pumps or pressurisation systems, or even by virtue of gravity.

The basal medium may in particular be added to the container vessel from a height above the cell-containing hydrogel fibre. This may be preferred when the cross-linking fluid is removed from the container vessel from a height below the cell-containing hydrogel fibre, for example at or near a bottom of the container vessel. When multiple hydrogels fibres are present in the container, it will be understood that basal medium may in particular be added to the container vessel from a height above some or all hydrogel fibres.

The differentiation medium may be added to the container vessel from a height above the cell-containing hydrogel fibre or all hydrogel fibres in the container vessel. In such embodiments, the differentiation medium may flow in a direction opposite to gravity. Alternatively, differentiation medium may be added to the container vessel from a height below the cell-containing hydrogel fibre or all hydrogel fibres in the container vessel.

It will be understood that while differentiation medium is added to the container vessel, other differentiation medium may also be removed from the container. As such, the differentiation medium may be refreshed, which may be required as the cells in the hydrogel fibres can extract contents such as nutrients from the differentiation medium, which may cause the differentiation medium from becoming at least partially depleted from these contents.

If the cells develop more into fat over time the scaffold might be floating up due to the difference of density between fat and water. If this occurs, the direction of the perfusion can be changed from top to bottom resulting in an optimal flow rate which would keep the scaffold in buoyancy.

Considering that the density of the hydrogel fibres may change over time, it may be preferred to change a direction of flow of differentiation medium in the container vessel accordingly. In embodiments, differentiation medium may thus be circulated through the container vessel, and a direction of the circulation may be based on a difference in density between the hydrogel fibre and the differentiation medium.

In particular, when the density of the hydrogel fibre exceeds the density of the differentiation medium, and the fibre thus sinks in the differentiation medium, it may be preferred to direct the flow of the differentiation medium in a direction opposite to gravity—i.e. upwards. As such, the flow of the differentiation medium may cause the fibre to be at least partially lifted, for example from a bottom of the container vessel or from a separator inside the container vessel. This in turn may increase the contact surface area between the fibre and the differentiation medium.

When the density of the differentiation medium exceeds the density of the hydrogel fibre, and the fibre thus floats in the differentiation medium, it may be preferred to direct the flow of the differentiation medium in the direction of gravity—i.e. downwards. As such, the flow of the differentiation medium may cause the fibre to be at least partially pushed downwards, for example from a ceiling of the container vessel or from a separator inside the container vessel. This in turn may increase the contact surface area between the fibre and the differentiation medium.

A third aspect provides a bioreactor for forming a cell-containing hydrogel fibre. The bioreactor may for example be used in a method according to the first aspect and/or the second aspect.

The bioreactor comprises a container vessel with an internal volume arranged for holding a volume of cross-linking fluid, an injector for injecting a flow of cell-containing hydrogel precursor fluid into the container vessel.

In a bioreactor according to third aspect, the injector may be arranged for injecting the flow of cell-containing hydrogel precursor fluid in a direction comprising an azimuthal direction component relative to the container vessel, in particular relative to at least part of an inner wall of the container vessel and/or the bioreactor may comprise a rotating motion inducer for inducing a rotating motion in the volume of cross-linking fluid held in the internal volume of the container vessel.

By virtue of a rotating motion induced in the volume of cross-linking fluid held in the internal volume of the container vessel, a leading end of a hydrogel fibre formed by injecting cell-containing hydrogel precursor fluid into the cross-linking fluid may be revolved at least partially around a vertical axis, in particular a centreline of the container vessel.

As an example, the rotating motion inducer may comprise a rotor, such as a propeller or impeller, arranged to rotate in the internal volume arranged for holding the volume of cross-linking fluid. As another example, the rotating motion inducer may be a stirring device, for example a magnetic stirrer.

In yet another example, the rotating motion inducer is comprised by a part of the bioreactor arranged for constituting a flow of fluid into the internal volume. When such a flow of fluid is injected into a volume of cross-linking fluid inside the internal volume, the kinetic energy of the flow of fluid may cause a rotating motion in the cross-linking fluid. The flow of fluid may be generally directed at a direction with an azimuthal or circumferential component.

In embodiments, the injector comprises an injection needle with a downstream end positioned in the internal volume of the container vessel. At an upstream end of the injection needle, the injection needle may be in fluid communication with a source of cell-containing hydrogel precursor fluid. In general, an injection needle may have an outlet diameter between 0.5 mm and 2 mm, for example approximately 1 mm, which may result in a hydrogel fibre with a similar diameter. It will be understood that other diameters may be used as well.

The injection needle may comprise a curved section at or near the downstream end of the injection needle. This may for example allow part of the injection needle upstream of the downstream end to be oriented generally vertically—e.g. generally parallel to a centreline of the container vessel—and the downstream end of the injection needle to be oriented generally horizontally.

When a section of the injector upstream of the curved section is oriented generally parallel to a centreline of the container vessel, the distal end of the injector may be positioned at a particular depth inside the internal volume. The section oriented generally parallel to the centreline of the container vessel is preferably radially offset from said centreline.

Preferably, the bioreactor comprises a plurality of injectors. With the plurality of injectors, more volume of hydrogel fibre may be formed in the same amount of time compared to using a single injector.

When the bioreactor comprises multiple injectors, downstream ends of the plurality of injectors may be positioned at different depths relative to the internal volume. This may prevent or reduce a chance of different fibres contacting and/or becoming entangled, in particular when the flows of cell-containing hydrogel precursor fluid are injected into the cross-linking fluid in a generally horizontal direction.

For example to further prevent or reduce a chance of different fibres contacting and/or becoming entangled, the bioreactor may further comprise a separator, positioned between downstream ends of two injectors of the plurality of injectors. It is generally preferred that a hydrogel fibre cannot pass through a separator or can move past a separator. Preferably, the separator is oriented generally horizontally.

For example to allow a flow of fluid, such as differentiation medium, through the separator, the separator may comprise one or more through-holes. The one or more through-holes may allow fluids in the internal volume, such as cross-linking fluid and/or differentiation medium and/or basal liquid, to move past the separator. Additionally or alternatively, one or more fluid passages may be present between the inner wall of the container vessel and the separator.

It is generally preferred to prevent a hydrogel fibre from passing through a through-hole of a separator. To prevent this from happening, or at least greatly reduce the chance of this from happening, a ratio between a cross-sectional area of the downstream end of the needle and a flow-through area of at least one of the through-holes may be 0.2 or more, in particular 0.25 or more. The cross-sectional area of the downstream end of the needle may generally define a cross-sectional shape of the fibre.

It may further be preferred to prevent the size of the flow-through area of the through-hole or through-holes from causing an excessive pressure drop for fluid such as differentiation medium flowing through the through-hole or through-holes.

A fourth aspect provides a bioreactor assembly for forming a cell-containing hydrogel fibre, comprising a bioreactor according to the third aspect. The bioreactor assembly may be used in a method according to the first and/or second aspect.

The bioreactor assembly further comprises a fluid container for holding a volume of cell-containing hydrogel precursor fluid and a gas source arranged for providing a pressurised gas. The fluid container may be in fluid communication with the injector of the bioreactor via a fluid conduit, and as such cell-containing hydrogel precursor fluid from the fluid container may be injected into the bioreactor via the injector.

The gas source of the bioreactor assembly may be in fluid communication with the fluid container via a gas conduit. As such, pressurised gas from the gas source may be used for expelling cell-containing hydrogel precursor fluid from the fluid container. It will be understood that also other means for transporting cell-containing hydrogel precursor fluid from the fluid container to the injector are envisioned, for example comprising one or more pumps and/or using gravity.

The bioreactor assembly may further comprise a three-way valve, positioned in the fluid conduit. When the bioreactor assembly comprises the three-way valve, the gas source may be in fluid communication with the injector of the bioreactor via the three-way valve and the fluid conduit. As such, gas from the gas source may be first used for flushing part of the fluid conduit downstream of the three-way valve, and subsequently the three-way valve may allow a flow of fluid from the fluid container, which is positioned upstream of the three-way valve, to the injector which is positioned downstream of the three-way valve.

In general, a hydrogel is a network wherein the discontinuous phase is solid and the continuous phase is water. The discontinuous phase is typically a network of hydrophilic polymer chains, which are crosslinked to form a three-dimensional network. Gels may be considered semi-solids and typically exhibit little to no flow at steady-state. The structural integrity of the hydrogel is typically not compromised by the presence of water. Hydrogels may for instance be capable of absorbing water to a high extent. Crosslinking (also referred to as gelation) of the polymer chains may be physical or chemical. Physical crosslinks for instance include ionic interactions, chain entanglement and hydrogen bonds. Chemical crosslinks are typically based upon covalent bonds between polymer chains.

The cell-containing hydrogel precursor fluid typically comprises cells preferably of mammal origin, in particular bovine origin. These cells may for instance be proliferated in an initial step and are subsequently ready to be seeded in order to differentiate into fat cells. It will however be understood that the methods and bioreactor disclosed herein may also be used in applications other than cultivation of fat, such as other pharmaceutical applications and/or tissue engineering. As such, also cell-containing hydrogel precursor fluid may comprise cells of human origin.

The cell-containing hydrogel precursor fluid further typically comprises a polysaccharide. Such polysaccharides may for instance include starch, chitin and/or alginate. Preferably the cell-containing hydrogel precursor fluid comprises alginate.

The cell-containing hydrogel precursor fluid is typically exposed to the cross-linking fluid to allow for crosslinking of the cell-containing hydrogel precursor fluid. The cell-containing hydrogel precursor fluid and the crosslinking fluid are typically low viscous liquids, for instance the viscosity may resemble the viscosity of water at ambient temperature (i.e. 20° C.). The cross-linking fluid is accordingly what provides the required conditions and/or components to allow for crosslinking. Crosslinking results in the formation of a cell-containing hydrogel.

The crosslinking process used in the present disclosure is typically sufficiently fast to allow for quick or preferably substantially instant crosslinking of the polymer chains. Typically, for the fast crosslinking process, the cross-linking fluid first contacts the outer surface of the cell-containing hydrogel precursor fluid flow. Accordingly, the outer layer of the cell-containing hydrogel precursor fluid flow may form a gel sufficiently fast to prevent the cell-containing hydrogel precursor fluid to spread out or lose its shape.

Preferably, the cross-linking fluid comprises an aqueous solution of calcium chloride, as this typically results in fast gelation of the cell-containing hydrogel precursor fluid. Typically, the concentration of the divalent cations is between 0.05 and 0.5 M.

### BRIEF DESCRIPTION OF THE FIGURES

In the figures,

FIG. 1 schematically shows an embodiment of a bioreactor assembly;

FIGS. 2A and 2B schematically depict an embodiment of a bioreactor, respectively in a longitudinal section view and a cross section view;

FIGS. 3A and 3B depict two particular options applicable separately to embodiments of the bioreactor;

FIGS. 4A and 4B schematically depict respectively in a longitudinal section view and a cross section view a further embodiment of a bioreactor

FIGS. 5A and 5B schematically depict respectively in a longitudinal section view and a cross section view another embodiment of a bioreactor; and

FIGS. 6A-6D schematically depict steps in a method of cultivating fat.

### DETAILED DESCRIPTION OF THE FIGURES

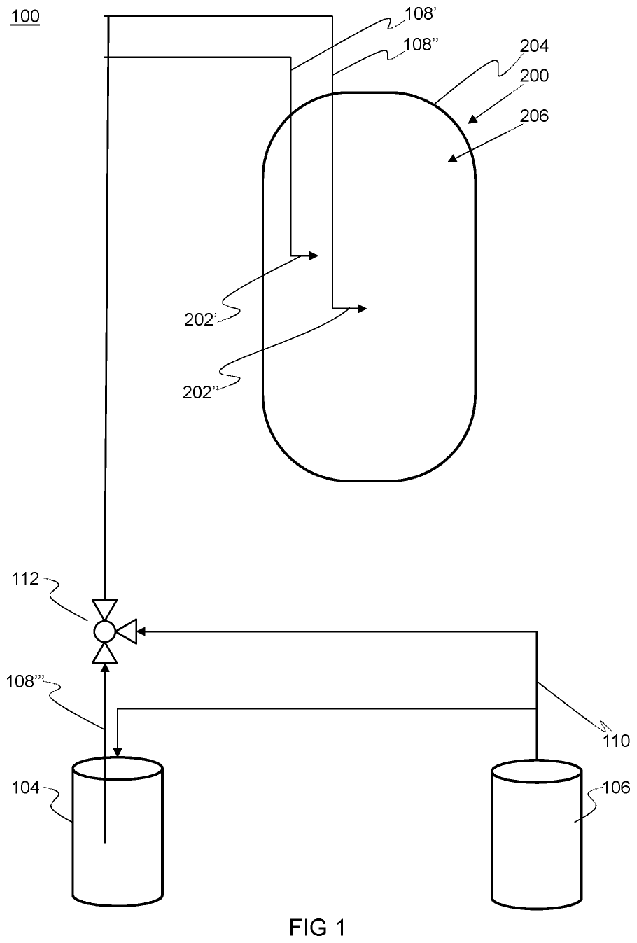

FIG. 1 schematically shows an embodiment of a bioreactor assembly 100. The bioreactor assembly 100 is shown comprising a bioreactor 200, which may generally be any embodiment of a bioreactor disclosed herein either explicitly or formed through a combination of features disclosed herein. The assembly 100 may be used for forming a cell-containing hydrogel fibre and/or for cultivating fat.

The bioreactor 200 comprises a container vessel 204 with an internal volume 206 arranged for holding a volume of cross-linking fluid and/or differentiation medium. Inside the bioreactor 200, in particular inside the internal volume 206, two injectors 202′, 202″ are depicted, which are arranged for injecting a flow of cell-containing hydrogel precursor fluid into the internal volume 206, and thus into cross-linking fluid provided inside the internal volume 206. It will be understood that bioreactors are envisioned comprising only one single injector, or more than two injectors.

The assembly 100 further comprises a fluid container 104 for holding a volume of cell-containing hydrogel precursor fluid. The fluid container 104 is in fluid communication with the bioreactor 200, in particular via a fluid conduit 108. As such, cell-containing hydrogel precursor fluid may be transported from the fluid container 104 into the bioreactor 200, in particular into the internal volume 206 of the container vessel 204, via the fluid conduit 108 and optionally any further conduit.

As a particular option depicted in FIG. 1, the bioreactor assembly 100 comprises a gas source arranged for providing a pressurised gas, here depicted as a gas canister 106. Alternatively, for example, a pump may be used as a gas source for providing pressurised gas. The pressurised gas may be used for expelling fluid from the fluid container 104. To this end, the gas canister 106 is in fluid communication with the fluid container 104 via a gas conduit 110.

It will be understood that any conduit, such as the fluid conduit of the gas conduit, may comprise any number of connected conduits, which may be directly connected or connected for example via one or more valves or pumps. A conduit, such as the fluid conduit of the gas conduit, may branch off into different conduits. For example, the fluid conduit 108 in FIG. 1 may comprises connected conduits 108′, 108″ and 108″.

FIG. 1 further depicts an option in which the bioreactor assembly 200 further comprises a three-way valve 112, which is positioned downstream of the fluid container and upstream of the bioreactor, and downstream of the gas canister 106. In a first state of the three-way valve, the gas canister 106 is in fluid communication with the bioreactor 200, in particular the internal volume 206, via at least part of the gas conduit 110, the three-way valve 112, and at least part of the fluid conduit 108. As such, in this first state, a pressurised gas may be transported through at least part of the fluid conduit 108, for example to flush said at least part of the fluid conduit 108.

In a second state of the three-way valve 112, the fluid container 104 is in fluid communication with the bioreactor 200, in particular the internal volume 206, via the fluid conduit 108, of which a part upstream of the three-way valve 112 is indicated with reference numeral 108″. In this second state, fluid from the fluid container 104 may be transported through at least part of the fluid conduit 108 to the bioreactor 200, for example after the at least part of the fluid conduit 108 has been flushed with gas.

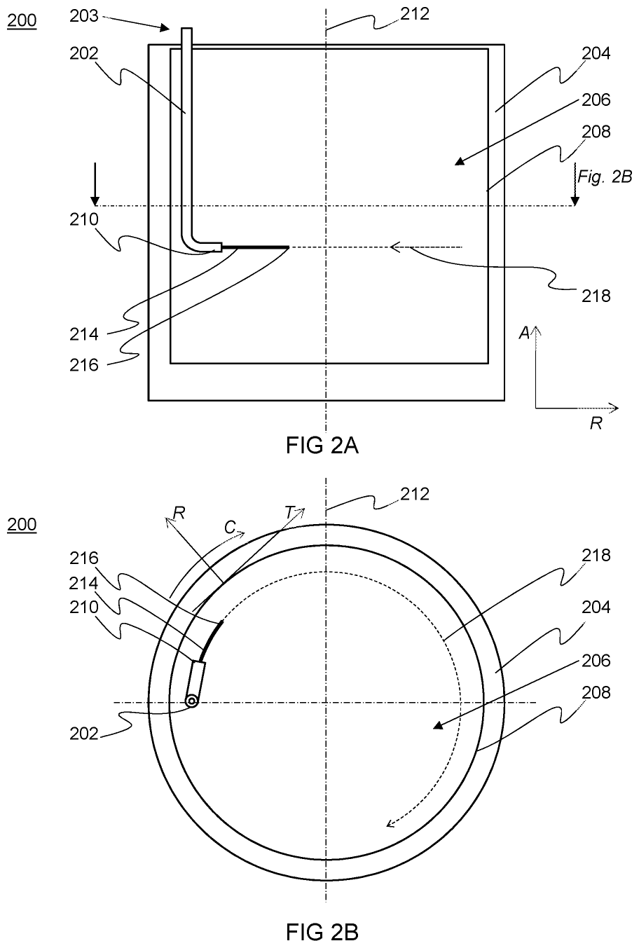

FIGS. 2A and 2B schematically depict an embodiment of a bioreactor 200 for forming a cell-containing hydrogel fibre, respectively in a longitudinal section view and a cross section view. The bioreactor 200 comprises the container vessel 204 with the internal volume 206 arranged for holding a volume of cross-linking fluid and/or differentiation medium. The internal volume 206 may be at least partially defined by an inner wall 208 of the container vessel 204. As schematically depicted in FIGS. 2A and 2B, at least part of the inner wall 208 can be curved, and the internal volume 206 may even be generally cylindrically shaped. In other embodiments, the internal volume 206 may have any other shape.

FIGS. 2A and 2B show the bioreactor 200 comprising an injector 202 for injecting a flow of cell-containing hydrogel precursor fluid into the internal volume 206 of the container vessel 204. At an upstream end 203, the injector 202 can be supplied with a flow of fluid, such as a flow of cell-containing hydrogel precursor fluid from a fluid conduit 108. At a downstream end, the injector 202 comprises an injection needle 210 which provides the outlet of the injector 202. In use, the outlet of the injection needle 210 may be submerged in the cell-containing hydrogel precursor fluid. A part of the injector 202 upstream of the outlet may be used for transporting fluid to the outlet end, in particular through the internal volume 206. In general, part of the injector 202 may be formed by part of the fluid conduit 108.

As a particular option depicted in FIG. 2A, the injector 202 comprises a substantially vertical section, which in use is oriented substantially vertically. Substantially vertically may be understood as at an angle of maximum +−45 degrees, or maximum +−20 degrees relative to the gravity vector, or even at an angle of maximum +−10 degrees, or even maximum +−5 degrees.

As can be seen for example in FIG. 2A, an injector 202 can enter the container vessel 204 from the top of the container vessel. This may for example eliminate or reduce a need for a fluid-tight seal between the container vessel and the injector and/or may prevent the need for a through-hole through a side wall and/or bottom of the container vessel. However, alternative embodiments of the bioreactor are envisioned wherein the injector 202 may enter the container vessel 204 from the bottom and/or through the side wall.

In general, the outlet of the injection needle 210 may be oriented at an angle relative to a section of the injector 202 upstream of the outlet of the injection needle 210. For example, the injector 202 may comprise a curved or bent section upstream of the outlet of the injection needle 210. This may allow the outlet of the injection needle 210 to be oriented substantially horizontal, as for example shown in FIG. 2A.

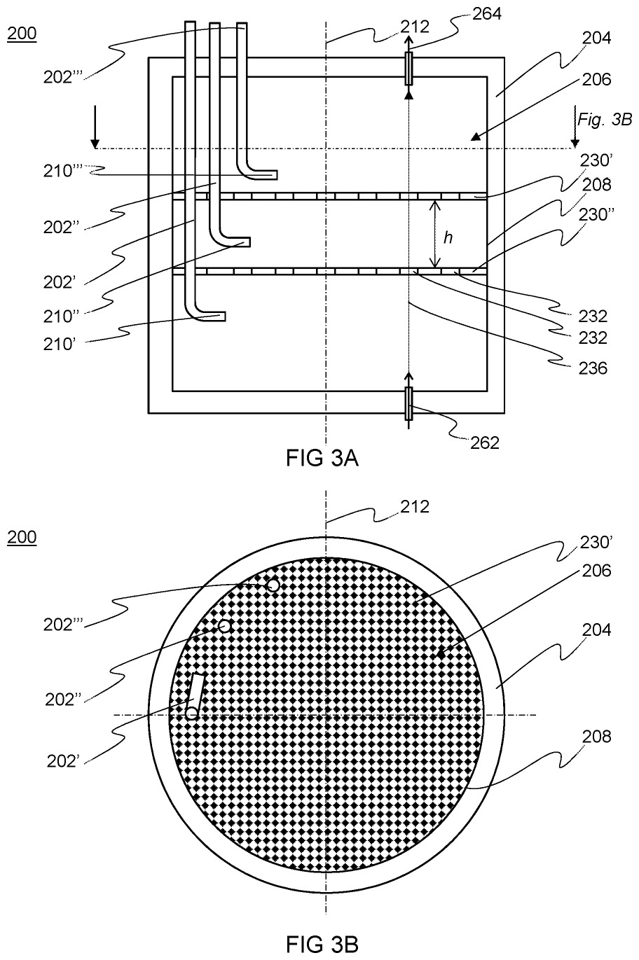

In FIGS. 2A and 2B, four directions are defined relative to the container vessel 204, and in particular relative to part of the inner wall 208 of the container vessel 204: an axial or longitudinal direction A, a radial direction R, a tangential direction T, and an azimuthal direction C. The same directions also apply in FIGS. 3A-6D.

FIGS. 2A and 2B show a cell-containing hydrogel fibre 214 being formed, by a flow of cell-containing hydrogel precursor fluid being injected into cross-linking fluid provided in the internal volume 206 of the container vessel 204. The hydrogel fibre 214 has a leading end 216. A typical path over which the leading end 216 may travel is schematically depicted in FIGS. 2A and 2B with a dashed arrow 218.

Preferably, for any method and bioreactor disclosed herein, the leading end of the hydrogel fibre revolves at least partially around the vertical axis during injection of the flow of hydrogel precursor fluid into the cross-linking fluid.

The length of a produced hydrogel fibre 214 may depend at least partially on the travel path 218 of the leading end 216, but may also be larger than a distance travelled by the leading end 216 inside the container vessel 204. It is envisioned that a single cell-containing hydrogen fibre may have any length, for example exceeding 20 cm, exceeding 50 cm, exceeding 1 metre, or even exceeding 2 metres.

As can be particularly seen in FIG. 2B, the travel path 218 for the leading end 216 of the hydrogel fibre 214 through the container vessel 204 can be a generally curved path 218, or at least comprise a curved path section. A curved path may be defined as any non-straight path. In particular, the path 218 revolves at least partially around a vertical axis, which in use may be the centreline 212 of the container vessel 204, or any other axis parallel to said centreline 212.

The path 218 depicted in FIG. 2B comprises an azimuthal direction component relative to the container vessel 204, in particular relative to at least part of the inner wall 208 of the container vessel. When the leading end 216 of the fibre moves over the path 218, the leading end 216 moves generally along the inner wall 208 of the container vessel.

FIGS. 3A and 3B schematically respectively in a longitudinal section view and a cross section view an embodiment of a bioreactor, with two particular options applicable separately to embodiments of the bioreactor 200, but which also may be combined.

As a first option, the bioreactor 200 comprises a plurality of injectors 202. This may allow for multiple cell-containing hydrogel fibres to be formed in the bioreactor 200, preferably simultaneously.

As depicted in FIG. 3A, the injection needles 210′, 210″, 210″ of the respective injectors 202′, 202″, 202′″ may be positioned at different heights in the internal volume 206 of the container vessel 204. Additionally or alternatively, injection needles may be radially and/or circumferentially spaced apart, as for example shown in FIG. 3B.

Alternatively or additionally to using a plurality of injectors 202, a single injector 202 may comprise multiple injection needles. The multiple injection needles may together have multiple outlets at which flows of cell-containing hydrogel precursor fluid may be injected into the container vessel, in particular at different locations-such that multiple cell-containing hydrogel fibres may be formed, preferably at least partially simultaneously.

Further alternatively or additionally to using a plurality of injectors 202, at least part of one or more injectors may be moveable inside the bioreactor. For example, an injection needle 210 may be moveable in the container vessel 204, in particular inside the internal volume 206. This may allow for a single injection needle 210 to be used to inject flows of cell-containing hydrogel precursor fluid into the cross-linking fluid at different location inside the internal volume 206.

An injection needle 210 may also, additionally or alternative, be moveable while a flow of cell-containing hydrogel precursor fluid is injected using said injection needle 210. For example, the injection needle 210 may be rotated about a substantially vertical axis. This may allow for the hydrogel fibre to be spread out more evenly and/or over a larger surface area and/or larger volume inside the bioreactor as the injection needle 210 may face in different direction by virtue of the movement and/or rotation of the injection needle. This in turn may reduce a chance of entangling and/or the amount of entanglement of the fibre may be reduced. The movement of the injection needle 210 may be an up-and-down movement, a see-sawing motion, a sweeping motion and/or a reciprocal movement.

As a second option, depicted in FIGS. 3A and 3B, the bioreactor 200 may comprise one or more separators 230′, 230″. In particular, the separators 230 each comprise one or more through-holes 232. Through said through-holes 232, a flow of fluid is allowed. In particular, a flow of cross-linking fluid, differentiation medium, and/or basal medium is allowed. Optionally, a flow of fat particles may also be allowed through said through-holes 232, for removing fat from the bioreactor 200. The fat particles may be formed by breaking up the hydrogel fibre, in particular the fat-containing hydrogel fibre, into smaller segments, for example by applying a shear force to the hydrogel fibre. It will be understood that for clarity and conciseness of the figures, not every through-hole 232 is provided with a reference numeral. In FIG. 3A, through-holes 232 are shown evenly spaced apart. In FIG. 3B, the separator 230′ is depicted as a sieve comprising a plurality of through-holes.

In general, the through-holes 232 of a separator 230 may restrict passage of objects with a cross-sectional dimension, such as a diameter, exceeding 1 mm or more, 2 mm or more, 4 mm or more, or even 6 mm or more.

A possible flow path for fluid through the container vessel 204, and through the separators 230′ 230″ is shown in FIG. 3A with a dotted arrow 236. The flow path 236 starts as an inlet flow 262 into the internal volume, passes through trough-holes 232 of the separators, and exits the internal volume 206 as an outlet flow 264. The direction of the flow 236 may in use be reversed.

Preferably, as shown in FIG. 3A, a separator 230 is positioned between outlets of two injection needles 210. As such, the separator 230 may prevent contact between cell-containing hydrogel fibres formed by injecting flows of cell-containing hydrogel precursor fluid from said two injection needles 210.

One or more of the separators 230 may in use be oriented substantially horizontally. As an option not shown in FIG. 3A, further separators 230 may be comprised by the bioreactor 200. For example, a separator may be present between a top side of the container vessel 204 and a highest of the injection needles. As such, contact between a hydrogel fibre formed by injecting fluid by the highest of the injection needles and an optional lid of the container vessel 204 may be avoided. As another example, a separator may be present between a bottom side of the container vessel 204 and a lowest of the injection needles. As such, contact between a hydrogel fibre formed by injecting fluid by the lowest of the injection needle and a bottom of the container vessel 204 may be avoided.

Adjacent separators 230′ 230″ may be separated by a particular height h, as indicated in FIG. 3A. For example, the height h may be between 1 cm and 10 cm, in particular between 3 cm and 8 cm, or even preferable approximately 5 cm.

In general, when an injection needle 210″ is positioned in-between two separators 230′, 230″, the injection needle 210″ may be positioned approximately centrally between the two separators 230′, 230″. However, it is also envisioned that the injection needle 210″ can be positioned closer to the upper one of the two separators 230′, or closer to the lower one of the two separators 230″. As such, for example, the injection needle 210″ may be positioned near the upper one of the two separators 230′, such that a distance between the injection needle 210″ and the upper one of the two separators 230′ is at least two times smaller than a distance between the injection needle 210″ and the lower one of the two separators 230″, or even at least four times smaller, or even at least ten times smaller.

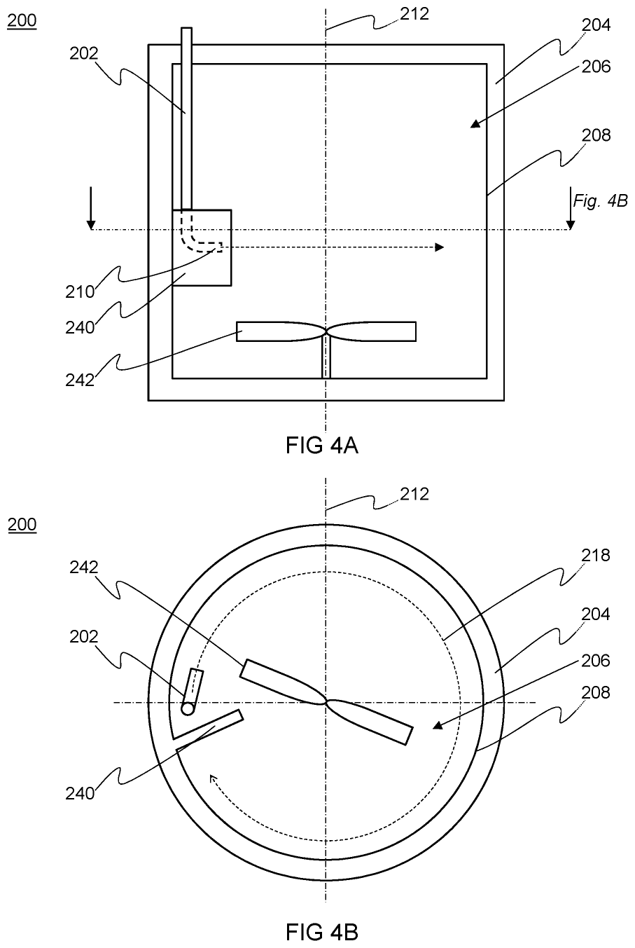

FIGS. 4A and 4B schematically depict respectively in a longitudinal section view and a cross section view a further embodiment of a bioreactor 200. The bioreactor 200 comprises a baffle 240, protruding generally radially into the internal volume 206. When the baffle 240 is positioned adjacent to the injection needle 210 of the injector 202, the baffle 240 may prevent a hydrogel fibre, in particular a leading end of a hydrogel fibre moving over the flow path 218, from coming near said injection needle 210. If the flow path 218 of the hydrogel fibre would position the hydrogel fibre, and in particular the leading end thereof, too close to the injection needle 210, the fibre may become entangled.

The baffle 240 may be oriented generally vertically in use. It will be understood that any embodiment of the bioreactor 200 disclosed herein may be provided with one or more baffles. The number of baffles may for example correspond to the number of the injection needles, such that each injection needle can be provided with an adjacent baffle. A baffle may extend between adjacent separators.

The baffle 240 may be rigidly connected to the bioreactor 200 or otherwise statically positioned inside the internal volume. Alternatively, the baffle 240 may be a moveable baffle. As such, the baffle may be moved in a generally azimuthal direction, for example by a sweeping or see-sawing motion in a generally horizontal plane. This movement of the baffle 240 may be used for agitating liquid inside the internal volume, which may result in a fibre 214 inside the internal volume from breaking into smaller segments. This in turn may allow fat formed by differentiation of cells in the cell-containing hydrogel fibre to be removed from the bioreactor more conveniently.

To move baffle 240, in particular to swivel the baffle 240 about the centreline 212 of the container vessel 204, the baffle may be connected to a shaft (not shown), which shaft may be rotated by a motor. The shaft may be oriented generally vertically, and the baffle may be connected to the shaft at a radius from the shaft. To prevent the baffle from contacting the injector 202, the path over which the baffle may be swivelled may be less than 360 degrees. The baffle may be moved over the path in a reciprocal manner. During movement, the baffle may remain oriented generally radially.

As a further option depicted in FIGS. 4A and 4B, which is as other options readily applicable to other embodiments of bioreactors, the bioreactor 200 comprises a rotatable propeller 242 arranged for inducing a rotating motion in a fluid held in the internal volume 206 of the container vessel 204. In the example of FIGS. 4A and 4B, a rotation axis of the propeller 242 is substantially aligned with the centreline 212 of the container vessel 204. However, in other envisioned embodiments, the rotation axis of the propeller 242 is generally substantially vertically. Although the propeller 242 is in FIG. 4A depicted near a bottom of the container vessel 204, the propeller 242—or more in general, the rotating motion inducer—may be positioned anywhere in the internal volume 206, also for example near a top of the container vessel 204.

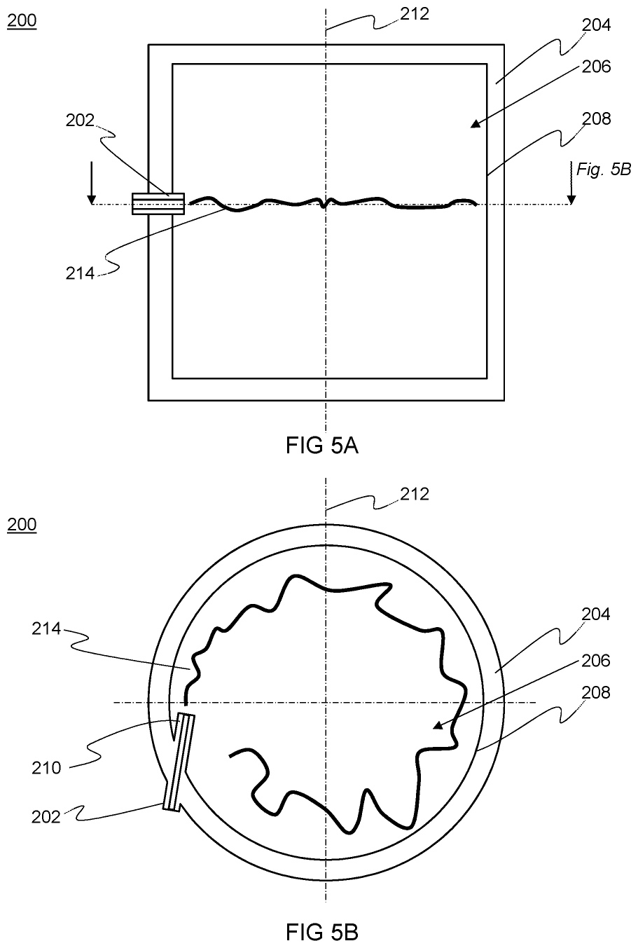

FIGS. 5A and 5B schematically depict respectively in a longitudinal section view and a cross section view another embodiment of a bioreactor 200, showing a particular example of an injector 202 which may be applied to any other embodiment of the bioreactor. In this particular embodiment, the injector 202 passes through a sidewall of the container vessel 204 of the bioreactor 200. As shown in FIG. 5B, the injection needle 210 may protrude into the internal volume 206 in which cross-linking fluid may be present. Alternatively, the injection needle 210 may have an outlet substantially flush with the inner wall 208 of the container vessel 204.

FIGS. 5A and 5B show an example of a final shape of the fibre 214. This shape may be obtained in the specific bioreactor disclosed in FIGS. 5A and 5B, but may also be obtained generally with any other embodiment of the bioreactor as disclosed herein. The final shape of the fibre 214 in general at least partially depends on the path travelled by the leading end of the fibre 214.

As depicted in FIG. 5A, the final orientation of the fibre 214 may be generally horizontal. However, different segments comprised by the fibre 214 may be oriented at an angle relative to the horizontal direction.

As depicted in FIG. 5B, an average or median final shape of the fibre 214 may be generally circular, or shaped as an arc—i.e. part of a circle. The average or median final shape of the fibre 214 generally revolves around a vertical axis, such as the centreline 212 of the container vessel 204. As can be seen in FIG. 5B, the final shape of the fibre 214 may not be a perfect circle, but may rather be formed by adjacent segments with different orientations, which segments together are generally revolved around the vertical axis.

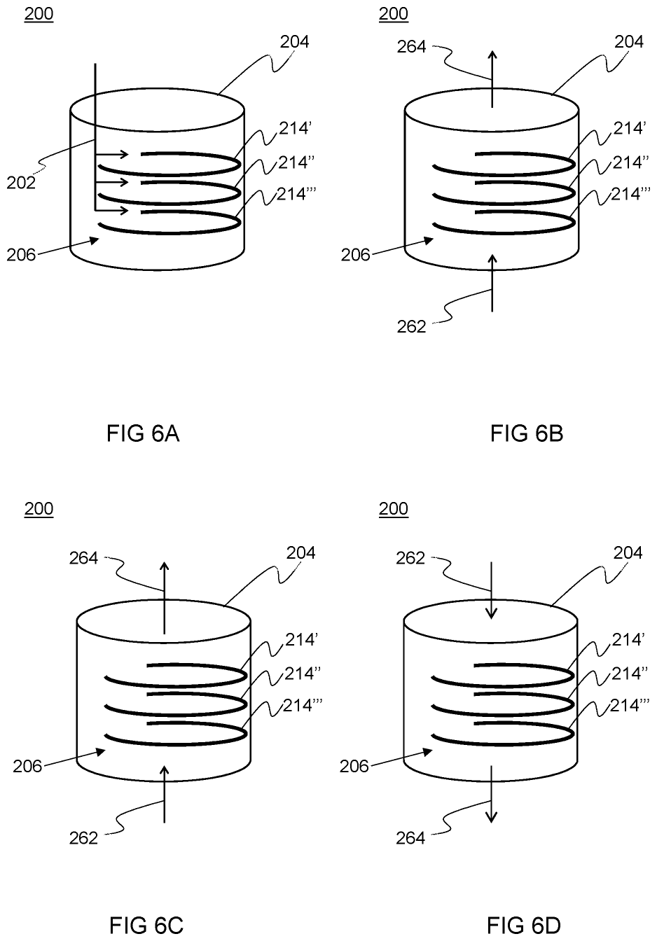

FIGS. 6A-6D schematically depict steps in a method of cultivating fat. In a first method step depicted in FIG. 6A, a plurality of cell-containing hydrogel fibres 214 has been formed inside the bioreactor 200. Although three distinct fibres 214′, 214″, 214″ have been depicted in FIG. 6A, it will be understood that in further embodiments of the method only a single fibre may have been formed, or any other number than three fibres may have been formed inside the bioreactor 200.

The fibres 214′, 214″, 214′″ may have been formed by injecting a flow of cell-containing hydrogel precursor fluid into cross-linking fluid present inside the bioreactor 200, using one or more injectors 202. In the state of the bioreactor 200 depicted in FIG. 6A, at least part of the internal volume 206 of the bioreactor 200 may hence be filled with cross-linking fluid. However, it will be understood that embodiments of the method of cultivating fat are also envisioned in which the fibres 214′, 214″, 214′″ are formed by a different method. For conciseness of the figures, the injector 202 is not depicted in FIGS. 6B-6D.

In an optional second method step, schematically depicted in FIG. 6B, the cross-linking fluid inside the bioreactor 200 is replaced with a basal medium. To this end, an inlet flow 262 into the internal volume 206 of the bioreactor 200 comprises basal medium. An outlet flow 264 out of the internal volume 206, and in particular out of the bioreactor 200, comprises the cross-linking fluid.

In a third method step, schematically depicted in FIG. 6C, the inlet flow 262 comprises differentiation medium, and the outlet flow 264 comprises one or more fluids, such as cross-linking fluid and/or basal medium. The outlet flow 264 may also comprise or consist of differentiation medium. As such, differentiation medium inside the internal volume 206 may be refreshed.

The inlet flow 262 is in FIG. 6C shown entering the internal volume 206 below the fibres 214′, 214″, 214′″. FIG. 6D shows an example wherein the inlet flow 262 enters the internal volume 206 above the fibres 214′, 214″, 214″. In general, although the inlet flow and outlet flow are depicted as passing through the bottom or lid of the bioreactor 200, it will be understood that at least part of the inlet flow and/or the outlet flow may pass through any other part of the bioreactor 200, such as a side wall of the container vessel 204, in any combination thereof.

When the inlet flow 262 enters the internal volume 206 below the fibres 214, the fibres may be subjected to an upwards force, which may cause the fibres to float or move upwards inside the differentiation medium inside the internal volume 206. When the inlet flow 262 enters the internal volume 206 above the fibres 214, in particular when the outlet flow 262 also removes fluid from the internal volume 206 from below the fibres 214, a downward force may prevent the fibres 214 to float, or at least reduce floatation of the fibres 214.

It has been observed that as the cells in the hydrogel fibres 214 differentiate into fat, the density of the hydrogel fibres 214 may decrease. This in turn may cause the hydrogel fibres 214 to float in the differentiation medium inside the internal volume 206. A downward force on the fibres 214 may be used to at least partially counteract the buoyancy force of the fibres 214.

Although in FIGS. 6B-6B a single inlet flow and a single outlet flow is depicted, it will be understood that conceivably multiple inlet flows and/or multiple outlet flows may be used for respectively supplying one or more fluids to the internal volume and removing one or more fluids from the internal volume, in any combination thereof, simultaneously or at separate instances.

As can be seen in FIGS. 6A-6D, throughout the steps of forming the cell-containing hydrogel fibre or fibres, replacing the cross-linking fluid in the container vessel 204 with a differentiation medium, and allowing differentiation of cells in the cell-containing hydrogel fibre into fat, the fibre of fibres may remain inside the container vessel 204, in particular inside the internal volume 206. As such, only a single container vessel 204 may be required, and it may be prevented that the fibre or fibres have to be moved between vessels.

In general, the flow of cell-containing hydrogel precursor fluid may be injected at a flow rate between 10 ml/min and 200 ml/min, in particular between 30 ml/min and 100 ml/min. Injection needles may be used with a diameter between 0.5 mm and 1.5 mm, in particular between 0.7 mm and 1.0 mm, for example depending on the desired diameter of the hydrogel fibre. The diameter of the container vessel, in particular of an internal volume of the container vessel, may be larger than 100 mm, larger than 200 mm, or even larger than 500 mm. A total volume of the internal volume of the container vessel may be 5 L or larger, 10 L or larger, or even 20 L or larger or even 50 L or larger. It will be understood that any combination of the values of the flow rate, diameter of the injection needle, diameter of the container vessel, and/or volume of the internal volume may be used.

### Example 1—Forming a Hydrogel Fibre

A cell-containing hydrogel fibre was formed by injecting a flow of cell-containing hydrogel precursor fluid comprising alginate into a cross-linking fluid comprising an aqueous solution of calcium chloride. A leading end of the hydrogel fibre revolved at least partially around a vertical axis, which resulted in a final form of the hydrogel fibre being revolved around said vertical axis. The cross-linking fluid was held in a generally cylindrical container vessel with a diameter of 210 mm and a total volume of 10 L.

The flow of cell-containing hydrogel precursor fluid was injected into the cross-linking fluid using an injection needle with a flow speed of about 55 ml/min. The injection needle had a diameter at the outlet of about 0.85 mm.

After the cell-containing hydrogel fibre had been formed, the cross-linking fluid in the container vessel has been replaced with a differentiation medium. Bovine proliferated cells inside the cell-containing hydrogel fibre differentiated into fat. Because the hydrogel fibre was revolved around the vertical axis, sufficient surface area of the hydrogel fibre was exposed to the differentiation medium allowing nutrients of the differentiation medium to reach the cells inside the fibre.

It is to be noted that the figures are only schematic representations of embodiments that are given by way of non-limiting examples. For the purpose of clarity and a concise description, features are described herein as part of the same or separate embodiments, in particular embodiments of the bioreactor and the bioreactor assembly. However, it will be appreciated that the scope of the disclosure may include embodiments having combinations of all or some of the features described.

1. A method of forming an hydrogel fibre inside a bioreactor, the method comprising steps of:
providing a container vessel of the bioreactor with a volume of cross-linking fluid therein; and
injecting a flow of hydrogel precursor fluid into the cross-linking fluid, whereby the hydrogel fibre is formed;
wherein a leading end of the hydrogel fibre revolves at least partially around a vertical axis, in particular at least partially around a centreline of the container vessel;
wherein the leading end of the hydrogel fibre revolves at least partially around the vertical axis at least through a 45 degrees section of a circumference/arc around said vertical axis, at least 90 degrees, at least 180 degrees, at least 270 degrees, or 360 degrees or more.

2. Method according to claim 1, wherein the hydrogel precursor fluid is a cell-containing hydrogel precursor fluid, comprising proliferated cells, in particular fibro-adipogenic progenitors, in particular of non-human mammalian origin, in particular of bovine origin. and the hydrogel fibre formed inside the bioreactor is a cell-containing hydrogel fibre.

3. Method according to claim 1, wherein the leading end of the hydrogel fibre is moved in the cross-linking fluid in a direction comprising a azimuthal direction component relative to the container vessel, in particular relative to at least part of an inner wall of the container vessel.

4. Method according to claim 1, wherein the flow of hydrogel precursor fluid is injected into the cross-linking fluid generally along an inner wall of the container vessel.

5. Method according to claim 1, wherein the flow of hydrogel precursor fluid is injected into the cross-linking fluid in a generally horizontal direction.

6. Method according to claim 1, wherein the flow of hydrogel precursor fluid is injected into the cross-linking fluid at least partially in a azimuthal direction relative to the container vessel, in particular relative to at least part of an inner wall of the container vessel.

7. Method according to claim 1, further comprising a step of inducing a rotating motion in the cross-linking fluid prior to and/or during the injecting of the flow of hydrogel precursor fluid into the cross-linking fluid, in particular a rotating motion around a substantially vertical axis.

8. Method according to claim 1, further comprising injecting an additional flow of hydrogel precursor fluid into the cross-linking fluid in the container vessel of the bioreactor, whereby an additional hydrogel fibre is formed, wherein the hydrogel fibre and the additional hydrogel fibre are separated by one or more separators of the bioreactor.

9. Method according to claim 8, wherein the flow of hydrogel precursor fluid and the additional flow of hydrogel precursor fluid are at least in part simultaneously injected into the cross-linking fluid.

10. Method according to claim 1, wherein the hydrogel precursor fluid is supplied from a fluid container, and the hydrogel precursor fluid is forced out of the fluid container using a pressurised gas.

11. (canceled)

12. (canceled)

13. Method of cultivating fat, comprising steps of:
forming a cell-containing hydrogel fibre inside a bioreactor using a method according to claim 2, to the extent dependent on claim 2;
replacing the cross-linking fluid in the container vessel with a differentiation medium;
allowing differentiation of cells in the cell-containing hydrogel fibre into fat; and
removing the fat from the container vessel.

14. (canceled)

15. (canceled)

16. (canceled)

17. (canceled)

18. (canceled)

19. (canceled)

20. (canceled)

21. Bioreactor for forming a cell-containing hydrogel fibre, the bioreactor comprising:
a container vessel with an internal volume arranged for holding a volume of cross-linking fluid and/or a differentiation medium;
an injector for injecting a flow of cell-containing hydrogel precursor fluid into the internal volume of the container vessel;
wherein:
the injector is arranged for injecting the flow of cell-containing hydrogel precursor fluid in a direction comprising an azimuthal direction component relative to the container vessel, in particular relative to at least part of an inner wall of the container vessel; and/or
the bioreactor comprises a rotating motion inducer for inducing a rotating motion in the volume of cross-linking fluid held in the internal volume of the container vessel.

22. Bioreactor according to claim 21, wherein the injector comprises an injection needle with an outlet positioned in the internal volume of the container vessel.

23. Bioreactor according to claim 22, wherein the outlet of the injection needle is oriented at an angle relative to a section of the injector upstream of the outlet of the injection needle.

24. Bioreactor according to claim 23, wherein a section of the injector upstream of the outlet of the injection needle is oriented generally parallel to a centreline of the container vessel.

25. Bioreactor according to claim 21, comprising a plurality of injection needles for injecting a plurality of flows of cell-containing hydrogel precursor fluid into the internal volume of the container vessel, preferably wherein downstream ends of the plurality of injection needles are positioned at different depths relative to the internal volume.

26. (canceled)

27. Bioreactor according to claim 21, further comprising a separator, positioned between downstream ends of two injection needles, preferably wherein the separator comprises one or more through-holes.

28. (canceled)

29. Bioreactor according to claim 27, wherein a ratio between a cross-sectional area of an outlet of the injector and a flow-through area of at least one of the through-holes is 0.2 or more, in particular 0.25 or more.

30. Bioreactor according to claim 21, further comprising a baffle protruding generally radially into the internal volume, wherein the baffle is positioned adjacent to the outlet of the injector, and an outlet of the injector is pointed in a direction away from the baffle.

31. (canceled)

32. Bioreactor assembly for forming a cell-containing hydrogel fibre, comprising:
a bioreactor according to claim 21;
a fluid container for holding a volume of cell-containing hydrogel precursor fluid;
a gas source arranged for providing a pressurised gas;
wherein
the fluid container is in fluid communication with the injector of the bioreactor via a fluid conduit;
the gas source is in fluid communication with the fluid container via a gas conduit;
a three-way valve, which is positioned downstream of the fluid container and upstream of the bioreactor; and
the gas source is in fluid communication with the injector of the bioreactor via the three-way valve and at least part of the fluid conduit.

33. (canceled)
